(19) United States
(12) Patent Application Publication
(10) Pub. No.: US 2022/0225946 A1
(43) Pub. Date: Jul. 21, 2022

(54) METHOD, DEVICE AND SYSTEM FOR OBTAINING VITAL SIGNAL

(71) Applicant: SHENZHEN DARMA TECHNOLOGY CO., LTD., Shenzhen City (CN)

(72) Inventor: Shaochun Zhuang, Shenzhen City (CN)

(21) Appl. No.: 17/577,303

(22) Filed: Jan. 17, 2022

(30) Foreign Application Priority Data
Jan. 18, 2021 (CN) 202110060042.4

(51) Int. Cl. A61B 5/00; A61B 5/11; A61B 5/339

(52) U.S. Cl. CPC A61B 5/7221; A61B 5/11; A61B 2562/0266; A61B 2562/0219; A61B 2562/0247; A61B 5/339

(57) ABSTRACT

A method for obtaining a vital signal, includes steps of: acquiring cardiac vibration signals of the subject through at least one vibration sensor placed under the back of the supine subject, and acquiring the ECG data of the subject synchronously; performing data validation testing on the ECG data and the cardiac vibration signals to generate a total validation duration of the ECG data and a total validation duration of the cardiac vibration signals; and determining an end of data acquisition based on the total validation duration of the ECG data and the total validation duration of the cardiac vibration signals.

### CROSS REFERENCE TO RELATED APPLICATIONS

The present invention is a 35 U.S.C. § 119 benefit of earlier filing date; right of priority of Chinese Application No. 202110060042.4, filed on Jan. 18, 2021, the disclosure of which is incorporated by reference herein.

### FIELD OF THE INVENTION

The present invention relates to the field of signal acquisition, and particularly relates to a method, device and system for obtaining a vital signal.

### BACKGROUND OF THE INVENTION

With the rapid development of science and technology, more and more products based on vibration sensing technology are applied to vital signs monitoring constantly emerging. Since the vibration sensor acquires the vibration signal, the micro-vibration of the environment, the body motion of the subject during the test, and the noise signal of the circuit itself will all interfere with the signal acquisition. The accuracy of the diagnostic results of a system for evaluating or monitoring a patient's cardiac function based on the vibration signal obtained by the vibration sensor depends on the validation of the obtained data, especially for a rapid evaluation, the data validation is more important. Therefore, for a rapid assessment of cardiac function, where the data acquisition time is short but the signal acquisition quality is high, a method, device and system for rapidly and high-quality acquisition of sign data are required.

### SUMMARY OF THE INVENTION

An object of the present invention is to provide a method, device and system for obtaining a vital signal, which aims to solve the problem that the vital signal needs to be acquired quickly and with high quality during a rapid assessment of cardiac function.

In a first aspect, the present invention provides a method for obtaining a vital signal, comprising steps of:

acquiring cardiac vibration signals through a vibration sensor placed under the back of a subject lying in the supine position, and acquiring the ECG data of the subject synchronously;

performing data validation testing on the ECG data and the cardiac vibration signals to generate a total validation duration of the ECG data and a total validation duration of the cardiac vibration signals; and

determining an end of data acquisition based on the total validation duration of the ECG data and the total validation duration of the cardiac vibration signals.

In a second aspect, the present invention provides a non-transitory computer-readable storage medium configured to store one or more computer programs including instructions that, when executed by at least one processor, cause the at least one processor to perform the steps of the above-mentioned method for obtaining a vital signal.

In a third aspect, the present invention provides a device for obtaining a vital signal, comprising: at least one processor; a memory; and one or more computer programs including instructions stored on the memory, and when executed by at least one processor, cause the at least one processor to perform the steps of the above-mentioned method for obtaining a vital signal.

In a fourth aspect, the present invention provides a system for obtaining a vital signal, comprising:

at least one vibration sensor, for acquiring cardiac vibration signals of the subject;

at least one ECG sensor, for acquiring ECG data of the subject; and

the above-mentioned device for obtaining a vital signal, connected to the at least one vibration sensor and the ECG sensor.

### Advantages

The method of the present invention can perform data validation testing while acquiring the ECG data and the cardiac vibration signals, then determine an end of data acquisition according to the total validation duration of the ECG data and the total validation duration of the cardiac vibration signals, output and display the total validation duration of the ECG data and the total validation duration of the cardiac vibration signals, which realizes controlling data validation in the data acquisition stage, and provides high-quality data for the subsequent rapid assessment of cardiac function.

### BRIEF DESCRIPTION OF THE DRAWINGS

FIG. 1 is a flowchart of a method for obtaining a vital signal in accordance with a first embodiment of the present invention;

FIG. 2 is a block diagram of a device for obtaining a vital signal in accordance with a third embodiment of the present invention; and

FIG. 3 is a block diagram of a system for obtaining a vital signal in accordance with a fourth embodiment of the present invention.

### DETAILED DESCRIPTION OF THE INVENTION

In order to make the objects, technical solutions, and advantages of the present invention clearer, the present invention will be further described in detail below in conjunction with the accompanying drawings and embodiments. It should be understood that the specific embodiments described here are only used to explain the present invention, but not to limit the present invention.

In order to illustrate the technical solutions of the present invention, the following is explained through specific embodiments.

### First Embodiment

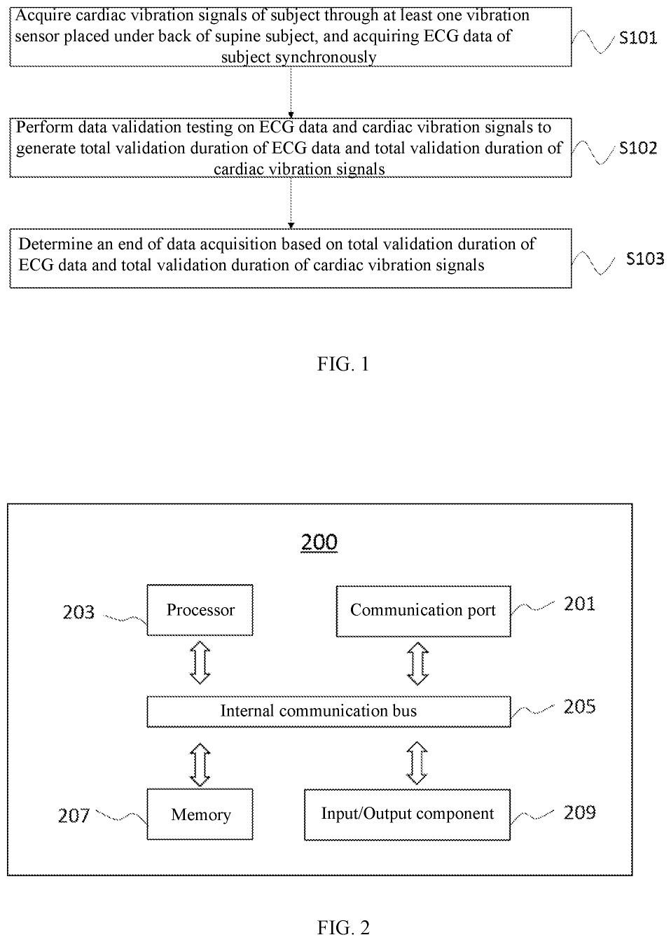

Referring to FIG. 1, a method for obtaining a vital signal provided in the first embodiment of the present invention comprises the following steps of S101 to S105. It should be noted that if substantially having the same results, the method for obtaining a vital signal of the present invention is not limited to the flowchart sequence shown in FIG. 1.

S101: acquiring cardiac vibration signals of the subject through at least one vibration sensor placed under the back of the supine subject, and acquiring ECG data of the subject synchronously.

In the first embodiment of the present invention, the at least one vibration sensor may be one or more of: an acceleration sensor, a velocity sensor, a displacement sensor, a pressure sensor, a strain sensor, or a sensor that converts physical quantities equivalently based on acceleration, velocity, pressure, or displacement (such as an electrostatic charge sensor, an inflatable pressure sensor, or a radar sensor, etc.), where the strain sensor may be a fiber-optic sensor.

In the first embodiment of the present invention, the subject may be in a supine position, with both legs naturally lying flat. Taking the subject in a supine position as an example, the vibration sensor may be configured to be placed under the subject's back on the medical bed. The cardiac vibration signals can be one or multiple signals, and correspondingly, at least one vibration sensor can be used. When there are multiple vibration sensors, they can be arranged along a height of the subject, and can also be arranged along the left and right directions of the human body.

The ECG data of the subject may be acquired through at least one connected lead such as electrode pads, ECG clips, or ECG balls, etc.

The ECG data and cardiac vibration signals are acquired synchronously on the time axis, a time delay correction can be performed according to a time delay of the system between different data (the ECG data and cardiac vibration signals) to meet the subsequent data processing, and it is also possible to perform a time correction during data processing to make the data in synchronization.

S102, performing data validation testing on the ECG data and the cardiac vibration signals to generate a total validation duration of the ECG data and a total validation duration of the cardiac vibration signals.

In the first embodiment of the present invention, S102 may include the following steps of S1021 to S1023.

S1021, performing cardiac cycle segmentation on waveforms of the ECG data and the cardiac vibration signals.

S1022, determining whether the ECG data is valid by cardiac cycle, and if it is valid, accumulating the corresponding cardiac cycle to the total validation duration of the ECG data.

Specifically, performing cardiac cycle segmentation on the waveform of the ECG data, and performing data validation testing on the waveform of each cardiac cycle. Where the data validation testing refers to analyzing the features of the data to be tested, and then determining whether the data to be tested matches a validation index, if yes, the data is valid, if not, the data is invalid. The step of performing data validation testing on the waveform of the ECG data in each cardiac cycle, may specifically be: performing morphological conformity judgment on the ECG data in each cardiac cycle. First, generating a quantifiable waveform quality assessment index based on one or more of: the shape, contour, amplitude, period, variability, etc. of the key feature points (e.g., P, Q, R, S, T, U waves) of the waveform of the ECG data in each cardiac cycle; then comparing the waveform quality assessment index with a preset standard ECG waveform quality index; if conforming to the validation comparison rule, determining the waveform in this cardiac cycle to be valid, and accumulate this cardiac cycle to the total validation duration of ECG data; if determine the waveform of the cardiac cycle to be invalid, this cardiac cycle will not be accumulated to the total validation duration of the ECG data. When the waveform quality assessment index adopts a numerical value, the validation comparison rule may be that: determine the waveform quality assessment index to be valid within a range of ±5% of the standard ECG waveform quality index, and vice versa. The waveform quality assessment index can also be further quantified by grades. The waveform quality assessment index of each cardiac cycle of ECG data can be set to A-level, B-level, C-level, and D-level according to the quality from high to low, and the standard ECG waveform quality index is A-level. When the quality assessment index of the waveform to be tested is determined above C-level, that is, including A-level, B-level, and C-level, it is determined as valid data, otherwise as invalid data. The ECG data may vary for individual, and in other embodiments of the present invention, the validation comparison rule for ECG data may also be adjusted according to the subject to be assessed.

S1023, determining whether the cardiac vibration signals are valid by cardiac cycle, and if it is valid, accumulating the corresponding cardiac cycle to the total validation duration of the cardiac vibration signals.

Since cardiac vibration signals are obtained through the vibration sensor, the disturbance of the surrounding environment or the subject's body motion will interfere with the signal acquisition. Therefore, before performing validation testing of cardiac vibration signals, performing cardiac vibration signals preprocessing. The preprocessing includes at least one of: filtering, noise removal and signal scaling; and specifically includes at least one of: HR (Infinite Impulse Response) filter, FIR (Finite Impulse Response) filter, wavelet filter, zero-phase bidirectional filter, polynomial smoothing filtering, integral transforms, and differential transforms, which is used to filter and denoise the cardiac vibration signals. The preprocessing may further include a step of: determining whether the cardiac vibration signals carrying power-line interference, and if yes, using a power frequency filter to remove power-line interference. In addition, the preprocessing can also include a step of removing high-frequency interference (for example, above 45 Hz).

The step of performing data validation testing on the cardiac vibration signals, further includes, but not limited to a step of: performing cardiac cycle segmentation on the waveform of the cardiac vibration signals, and performing validation testing on the waveform of each cardiac cycle, such as performing morphological conformity judgment on the waveform. First, generating a quantifiable waveform quality assessment index based on one or more of: the shape, contour, amplitude, period, variability, etc. of the key feature points (such as H wave, I wave, J wave, K wave, L wave, M wave, N wave), then comparing the waveform quality assessment index with a preset standard cardiac vibration waveform quality index: if conforming to the validation comparison rule, determining the waveform of this cardiac cycle to be valid, and accumulating this cardiac cycle to the total validation duration of the cardiac vibration signals; if determining the waveform of this cardiac cycle to be invalid, not accumulating this cardiac cycle to the total validation duration of cardiac vibration signals. The validation comparison rule may be: determining the waveform quality assessment index to be valid within a range of ±5% of the standard cardiac vibration waveform quality index, and vice versa. The waveform quality assessment index can also be further quantified by grades. The waveform quality assessment index of each cardiac cycle of the cardiac vibration signals can be set to A-level, B-level, C-level, and D-level according to the quality from high to low. The standard cardiac vibration waveform quality index is A-level. When the waveform quality assessment index to be tested is determined above the C-level, that is, including the A-level, B-level, and C-level, it is determined as valid data; otherwise, it is invalid. The cardiac vibration signals may vary for individual, in other embodiments of the present invention, the validation comparison rule of cardiac vibration signals can also be adjusted according to the condition (such as age, weight, presence or absence of underlying diseases, etc.) of the object to be assessed.

In the first embodiment of the present invention, steps S1022 and S1023 may be performed in parallel or simultaneously. Data validation testing of cardiac vibration signals and ECG data may be performed in real time. When acquiring the cardiac vibration signals and ECG data of the next cardiac cycle, data validation testing can be performed on the acquired cardiac vibration signals and ECG data of the current cardiac cycle at the same time.

S103, determining an end of data acquisition based on the total validation duration of the ECG data and the total validation duration of the cardiac vibration signals.

The step of determining an end of data acquisition based on the total validation duration of the ECG data and the total validation duration of the cardiac vibration signals, specifically, when the total validation duration of the ECG data is greater than or equal to a set validation duration threshold, and the total validation duration of the cardiac vibration signals is also greater than or equal to a set validation duration threshold, the data acquisition is qualified, and the data acquisition can be ended. For example, for a rapid assessment of cardiac function, a validation duration threshold can be set as 60 or 90 seconds, etc. When a total validation duration of ECG data and a total validation duration of cardiac vibration signals are both greater than or equal to 90 seconds, stop data acquisition.

In the first embodiment of the present invention, basic parameters such as age, gender, height, weight, etc. of the subject can also be collected before the test and are used as parameters for setting the validation duration threshold, so that the validation duration threshold can be changed according to the test environment and subjects.

In some embodiments of the present invention, a method for obtaining a vital signal may further include the following steps:

S104, outputting the total validation duration of the ECG data and the total validation duration of the cardiac vibration signals to an output device. The total validation duration of the ECG data and the total validation duration of the cardiac vibration signals can be displayed by means of but not limited to: a direct cumulative time, a progress bar, and a pie chart, etc. For example, when the total validation duration of ECG data and the total validation duration of cardiac vibration signals are output to a display, a first timer and a second timer can be respectively used to display the total validation duration of ECG data and the total validation duration of cardiac vibration signals. After performing data validation testing on the ECG data and the cardiac vibration signals at step S102, updating a total validation duration of the ECG data and a total validation duration of the cardiac vibration signals.

Specifically, after determining an end of data acquisition, a total validation duration of ECG data and a total validation duration of cardiac vibration signals can be displayed on a user interactive device (such as tablet computer, mobile phone, touchable display, etc.), and prompt information can also be sent to the subject to remind the acquisition end and removing the ECG acquisition equipment from the subject. For example, prompt by displaying “the validation duration of the acquired data has reached the standard, please end the acquisition” or other similar expressions on the GUI interface through a pop-up window display, or prompt by playing a sound such as “acquisition ends” or other similar words, or prompt by playing a tone of beep, beep, beep or other sound to indicate that the acquisition can be ended; flashing lights, marquee lights, completion of the progress bar, completion of the pie chart, etc. can also be used to indicate that the acquisition can be ended. In some embodiments of the present invention, the ECG data acquisition duration and the cardiac vibration signals acquisition duration can also be displayed on the user interactive GUI interface by means of, but are not limited to direct cumulative time, progress bar, pie chart, etc.

S105, outputting ECG data and cardiac vibration signals to a display device for displaying waveforms of the same. For example, the waveforms of the ECG data and the cardiac vibration signals can be displayed on a handheld device, and can be real-time output, so that the collector (doctor, nurse, or caretaker, etc.) can observe the waveform quality of the acquired data in real time. Further, the waveform quality index of the ECG data of each cardiac cycle and the waveform quality index of the cardiac vibration signals of each cardiac cycle can also be output to a display device for display, so as to provide a reference for the collector. The step S105 may be performed in parallel (simultaneously) with the step S104.

In the first embodiment of the present invention, the method can further comprise steps of:

performing data interference analysis on the ECG data, specifically, performing waveform morphological analysis on the ECG data, and determining whether the ECG data carries power-line interference, or whether a connected lead is disconnected, and generating ECG signal interference prompt information and outputting it by means of a display device or a sound device; and

performing waveform morphological analysis on the cardiac vibration signals, determining whether there is body motion interference, or whether there is a subject on the vibration sensor, or whether there is muscle movement interference, and then generating a cardiac vibration signal interference prompt information.

The ECG signal interference prompt information and the cardiac vibration signal interference prompt information can be outputted by means of a display device or a sound device. For example, prompt by displaying “ECG lead off, please reconnect”, “cardiac vibration signals is disturbed by body motion, please keep still” or other similar expressions on the GUI interface through the pop-up window displays, or play a prompt sound when a lead is off.

### Second Embodiment

The second embodiment of the present invention provides a non-transitory computer-readable storage medium configured to store one or more computer programs including instructions that, when executed by at least one processor, cause the at least one processor to perform the steps of the method for obtaining a vital signal in the first embodiment.

### Third Embodiment

The third embodiment of the present invention provides a device 200 for obtaining a vital signal. Referring to FIG. 2, the device 200 may be a special purpose computer device specially designed to process vibration information from a vibration sensor.

For example, the device 200 may comprises: a communication port 201 connected to a network for communication; and at least one processor 203 for executing computer instructions. The computer instructions may comprise, for example, routines, programs, objects, components, data structures, processes, modules, or functions etc. for executing the method for obtaining a vital signal described in the first embodiment of the present invention. For example, the processor 203 can obtain cardiac vibration signals acquired through the vibration sensor and obtain the ECG data through an ECG device, and perform data validation testing on the cardiac vibration signals and the ECG data.

In some embodiments, the at least one processor 203 may include one or more of hardware processors, for example, any circuit or processor capable of performing one or more functions, or any combination thereof, such as microprocessors, Reduced Instruction Set Computers (RISCs), Application Specific Integrated Circuits (ASICs), Graphics Processing Units (GPUs), Central Processing Units (CPUs), Digital Signal Processors (DSPs), Field Programmable Gates Array (FPGA), Advanced RISC Machine (ARM), Programmable Logic Device (PLD), etc.

The device 200 may further comprise: an internal communication bus 205 for internal communication, a memory 207 configured for storing data and instructions thereon, and program instructions stored in memory 207 in other types of non-transitory storage media, and can be executed by the processor 203. The methods and/or processes of the present application may be implemented by program instructions. The device 200 can further comprise an input/output component 209 for data input/output. For example, a subject or collector can use an input device (e.g., a keyboard, or a touch screen, etc.) to input some data into the device 200 through the input/output component 209, such as the subject's age, gender, height, weight, etc. The device 200 may also output data to an output device (e.g., a display, or a printer, etc.) through the input/output component 209.

For the convenience of description, only one processor is described in the device 200 for obtaining a vital signal in the present invention. However, it should be noted that the device 200 may also include multiple processors, and thus, the process and/or method disclosed in the present invention may be performed by one processor as described above, or may be executed jointly by multiple processors. For example, if the processor 203 of the device 200 performs steps A and B in the present application, steps A and B may also be performed jointly or separately by two different processors (e.g., the first processor executes step A, the second processor executes step B, or the first and second processors jointly execute steps A and B).

### Fourth Embodiment

The fourth embodiment of the present invention provides a system for obtaining a vital signal, comprising:

at least one vibration sensor, for acquiring cardiac vibration signals of the subject;

at least one ECG sensor, for acquiring ECG data of the subject; and

the device for obtaining a vital signal provided in the third embodiment of the present invention, connected to the at least one vibration sensor and the at least one ECG sensor.

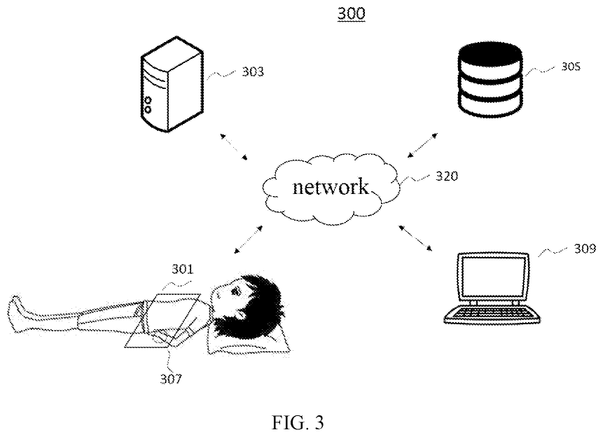

Referring to FIG. 3, a system 300 for obtaining a vital signal may comprise, but not limited to: one or more vibration sensors 301, one or more ECG sensors 307, one or more devices 303 for obtaining a vital signal, one or more storage devices 305, and one or more output devices 309.

Where the vibration sensor 301 can be an acceleration sensor, a velocity sensor, a displacement sensor, a pressure sensor, a strain sensor, a stress sensor, and can also be a sensor that converts physical quantities equivalently based on acceleration, velocity, displacement, or pressure (such as an electrostatic charge sensor, an inflatable pressure sensor, or a radar sensor, etc.), where the strain sensor may be a fiber-optic sensor. The vibration sensor may not be in direct contact with the subject, for example, when the vibration sensor is a fiber optic sensor, the fiber optic sensor may be placed under the back of a lying supine subject, where the fiber optic sensor may be arrayed.

The ECG sensor 307 is used to acquire the ECG data of the subject, and can be configured as an ECG clip, which is clamped on the wrist of the subject to acquire ECG data, as shown in FIG. 3. The ECG sensor 307 can also be configured as an ECG ball. When the subject is lying on his back, his hands can be placed on the ECG ball to acquire ECG data. The ECG sensor 307 can also be configured as an ECG patch, which is attached to the subject's skin to acquire ECG data. The ECG sensor 307 can be configured as a handheld ECG device, which the subject holds with both hands to acquire ECG data. The ECG sensor 307 may be a single-lead sensor, a twelve-lead sensor, or the like.

The device 303 for obtaining a vital signal as described in the third embodiment of the present invention, can be connected to the vibration sensor 301 and the ECG sensor 307 through the network 320. The network 320 may be a single network, such as a wired network or a wireless network, or a combination of multiple networks. The network 320 may include, but not limited to, a local area network, a wide area network, a public network, a private network, or the like. The network 320 may include various network access points, such as wireless or wired access points, base stations or network access points, through which other components of the system 300 can connect to the network 320 and transmit information through the network. The device 303 for obtaining a vital signal may also be connected to the vibration sensor 301 and the ECG sensor 307 through data transmission lines.

The storage device 305 may be configured to store data and instructions. The storage device 305 may include, but not limited to, Random Access Memory, Read Only Memory, Programmable Read Only Memory, or the like. The storage device 305 may be a device that stores information by means of electrical energy, magnetic energy, or optical means, such as hard disks, floppy disks, magnetic core memories, CDs, DVDs, or the like.

In some examples, the system 300 for obtaining a vital signal may further include an output device 309, and the output device 309 is configured to output the information after the data acquisition is ended, and the output means includes but are not limited to graphics, text, data, or voice, etc. For example, output the information after the data acquisition by one or more of: graphic display, digital display, voice announcement, and braille display, etc. The output device 309 may be one or more of: a display, a mobile phone, a tablet computer, a projector, a wearable device (watch, headset, glasses, etc.), a braille display, and the like. In some embodiments, the output device 309 can be used to display the waveforms of the subject's ECG data and cardiac vibration signals in real time. The output device 309 can also be used to implement a prompting function, for example, play a voice prompt. When the ECG connection to the subject is faulty and there is no ECG signal, or the subject's lying position deviates, the subject or the collector can be prompted by voice to check and reconnect the ECG connection, and the subject can adjust his position.

The method of the present invention can perform data validation testing on the ECG data and the cardiac vibration signals while acquire the ECG data and the cardiac vibration signals, then determine an end point of data acquisition according to the data validation duration, and output and display the validation duration of the ECG data and the validation duration of the cardiac vibration signals, which realizes controlling data validation duration in the data acquisition stage, and provides high-quality data for signal processing required for a subsequent rapid assessment of cardiac function.

A person of ordinary skill in the art can understand that all or part of the steps in the various methods of the above-mentioned embodiments can be completed by a program instructing relevant hardware. The program can be stored in a computer-readable storage medium. The computer-readable storage medium may comprise: ROM (Read Only Memory), RAM (Random Access Memory), magnetic disk or optical disk, etc.

The foregoing descriptions are only preferable embodiments of the present invention, and are not intended to limit the present invention. Any modification, equivalent replacement, and improvement made within the spirit and principle of the present invention shall be included in the protection scope of the present invention.

What is claimed is:

1. A method for obtaining a vital signal, comprising steps of:
acquiring cardiac vibration signals of a subject through at least one vibration sensor placed under the back of the subject lying in a supine position, and acquiring the ECG data of the subject synchronously;
performing data validation testing on the ECG data and the cardiac vibration signals to generate a total validation duration of the ECG data and a total validation duration of the cardiac vibration signals; and
determining an end of data acquisition based on the total validation duration of the ECG data and the total validation duration of the cardiac vibration signals.

2. The method of claim 1, wherein the at least one vibration sensor is one or more of: an acceleration sensor, a velocity sensor, a displacement sensor, a pressure sensor, a strain sensor, a stress sensor, and a sensor that converts physical quantities equivalently based on acceleration, velocity, displacement, or pressure.

3. The method of claim 2, wherein the strain sensor is a fiber-optic sensor.

4. The method of claim 1, wherein the step of performing data validation testing on the ECG data and the cardiac vibration signals to generate a total validation duration of the ECG data and a total validation duration of the cardiac vibration signals, further comprises steps of:
performing cardiac cycle segmentation on waveforms of the ECG data and the cardiac vibration signals;
determining whether the ECG data is valid by cardiac cycle, and if it is valid, accumulating the corresponding cardiac cycle to the total validation duration of the ECG data; and
determining whether the cardiac vibration signals is valid by cardiac cycle, and if it is valid, accumulating the corresponding cardiac cycle to the total validation duration of the cardiac vibration signals.

5. The method of claim 4, wherein the step of determining whether the ECG data is valid by cardiac cycle, specifically is: performing morphological conformity judgment on the ECG data in each cardiac cycle, generating a quantifiable waveform quality assessment index based on one or more of: shape, contour, amplitude, period, variability of key feature points of the waveform; then comparing the waveform quality assessment index with a preset standard ECG waveform quality index;
the step of determining whether the cardiac vibration signals is valid by cardiac cycle, specifically is: performing morphological conformity judgment on the cardiac vibration signals in each cardiac cycle, generating a quantifiable waveform quality assessment index based on one or more of: shape, contour, amplitude, period, variability of key feature points of the waveform; then comparing the waveform quality assessment index with a preset standard cardiac vibration waveform quality index.

6. The method of claim 1, further comprising a step of:
outputting the total validation duration of the ECG data and the total validation duration of the cardiac vibration signals to an output device.

7. The method of claim 6, wherein the step of outputting the total validation duration of the ECG data and the total validation duration of the cardiac vibration signals to an output device, specifically is:
outputting the total validation duration of the ECG data and the total validation duration of the cardiac vibration signals to a display device, where a display means is one or more of: a direct cumulative time display, a progress bar display, and a pie chart display.

8. The method of claim 1, further comprising a step of:
outputting the total validation duration of the ECG data and the total validation duration of the cardiac vibration signals to a display device for waveform display.

9. The method of claim 1, wherein the step of determining an end of data acquisition based on the total validation duration of the ECG data and the total validation duration of the cardiac vibration signals, specifically is:
when the total validation duration of the ECG data and the total validation duration of the cardiac vibration signals are both greater than or equal to a set validation duration threshold, the data acquisition ends.

10. The method of claim 1, further comprising a step of:
generating an ECG signal interference prompt and/or a cardiac vibration signal interference prompt based on the total validation duration of the ECG data and/or the total validation duration of the cardiac vibration signals.

11. A non-transitory computer-readable medium having one or more computer programs including instructions stored thereon, which when being executed by one or more processor, cause the one or more processor to perform the method of claim 1.

12. A device for obtaining a vital signal, comprising:
one or more processor;
a memory; and
one or more computer programs including instructions;
wherein the one or more processor and the memory are connected by a bus; the one or more computer programs including instructions are stored on the memory, and which when being executed by the one or more processor, cause the one or more processor to perform a method for obtaining a vital signal, comprising steps of:
acquiring cardiac vibration signals of a subject through at least one vibration sensor placed under the back of the subject lying in a supine position, and acquiring the ECG data of the subject synchronously;
performing data validation testing on the ECG data and the cardiac vibration signals to generate a total validation duration of the ECG data and a total validation duration of the cardiac vibration signals; and
determining an end of data acquisition based on the total validation duration of the ECG data and the total validation duration of the cardiac vibration signals.

13. A system for obtaining a vital signal, comprising:
at least one vibration sensor, for acquiring cardiac vibration signals of a subject;
at least one ECG sensor, for acquiring ECG data of the subject; and
a device for obtaining a vital signal;
wherein the device is connected to the at least one vibration sensor and the at least one ECG sensor; and the device comprises:
one or more processor;
a memory; and
one or more computer programs including instructions;
wherein the one or more processor and the memory are connected by a bus; the one or more computer programs including instructions are stored the memory, and which when being executed by the one or more processor, cause the one or more processor to perform a method for obtaining a vital signal, comprising steps of:
acquiring cardiac vibration signals of the subject through the at least one vibration sensor placed under the back of the subject lying in a supine position, and acquiring the ECG data of the subject synchronously;
performing data validation testing on the ECG data and the cardiac vibration signals to generate a total validation duration of the ECG data and a total validation duration of the cardiac vibration signals; and
determining an end of data acquisition based on the total validation duration of the ECG data and the total validation duration of the cardiac vibration signals.

14. The system of claim 13, further comprising:
a display device, for displaying one or more of: a waveform of the ECG data, a waveform of the cardiac vibration data, the total valid duration of the ECG data, the total valid duration of the cardiac vibration data, an ECG signal interference prompt, or a cardiac vibration signal interference prompt.
